(19) United States
(12) Patent Application Publication
(10) Pub. No.: US 2006/0073156 A1
(43) Pub. Date: Apr. 6, 2006

(54) Fosfomycin and n-acetylcysteine for the treatment of biofilms caused by escheric ia coli and other pathogens of the urinary tract

(75) Inventors: Domenico Ungheri, Milano (IT); Luciano Licciardello, Milano (IT); Giovanni Battista Colombo, Milano (IT)

Correspondence Address: OBLON, SPIVAK, MCCLELLAND, MAIER & NEUSTADT, P.C., 1940 DUKE STREET, ALEXANDRIA, VA, 22314, US

(73) Assignee: ZAMBON GROUP S.P.A., VICENZA (IT)

(21) Appl. No.: 10/526,404

(22) PCT Filed: Sep. 1, 2003
(86) PCT No.: PCT/EP03/09742

§ 371 Date: Sep. 13, 2005

(30) Foreign Application Priority Data
Sep. 4, 2002 (IT) MI2002A 001881

(51) Int. Cl. A61K 39/00; A61K 39/38

(52) U.S. Cl. 424/184.1

(57) ABSTRACT

Pharmaceutical compositions for the treatment of infections by pathogens of the urinary tract and, more particularly, compositions containing fosfomycin optionally associated with N-acetylcysteine.

The present invention relates to pharmaceutical compositions for the treatment of infections by pathogens of the urinary tract and, more particularly, it relates to compositions containing fosfomycin optionally associated with N-acetylcysteine.

N-acetylcysteine (hereinafter NAC) (The Merck Index XIII Ed., No. 90, page 94) is a known drug having mucolytic and antioxidant activity which is sold, for example, in Italy under the trademark FLUIMUCIL.

Fosfomycin (The Merck Index XIII Ed., No. 4277, page 4281) is a known drug having antibiotic activity which is sold in Italy as trihydroxymethylaminomethane salt under the trademark MONURIL. Various pathogens of the urinary tract, for example Escherichia coli, have the ability to produce an abundant extracellular mucopolysaccharide compound that promotes bacterial adhesion and the formation of biofilms.

Biofilm has been shown to be a primary virulence factor in the infections caused by these microorganisms.

The biofilm often enables bacteria to colonize external surfaces such as prostheses or catheters.

Inside the biofilm, the bacteria can grow in an environment that is protected both against the immune responses and against antibiotics.

Biofilms are therefore an important problem in antibiotic therapy and are often the cause of recurrent infections.

It will therefore be apparent that there is a need for substances that are capable of inhibiting the formation of biofilms or disrupting existing biofilms.

NAC has been recognized as possessing the ability to disrupt biofilms of P. aeruginosa [Antimicrob. Agents Chemother., 35, 1258, (1991)] and to inhibit their formation by S. epidermidis [J. Ant. Chem., 39, 643, (1997)].

We have now found that fosfomycin is capable of decreasing the amount of biofilms formed by pathogens.

We have also found that NAC is able to increase the activity of fosfomycin in inhibiting the formation of or disrupting biofilms of pathogens. In particular of pathogens of the urinary tract such as Escherichia coli.

From this point forward we shall focus on Escherichia coli, it being understood that what is stated, also applies to other pathogens of the urinary tract that are capable of producing biofilms.

Therefore, it is a first object of the present invention to provide a process for preparing a pharmaceutical composition containing fosfomycin for the preventive or disruptive treatment of biofilms produced by Escherichia coli and by other pathogens of the urinary tract.

Moreover, it is a second object of the present invention to provide a process for preparing a pharmaceutical composition containing NAC that can be used for increasing the activity of fosfomycin in inhibiting or disrupting the biofilms produced by Escherichia coil and by other pathogens of the urinary tract.

Additionally, it is a third object of the present invention to provide a pharmaceutical composition containing fosfomycin and NAC and the use thereof to inhibit or disrupt biofilms produced by Escherichia coil and by other pathogens of the urinary tract.

Finally, it is a fourth object of the present invention to provide a process for preparing a pharmaceutical composition containing NAC that can be used for inhibiting or disrupting biofilms produced by Escherichia coli and by other pathogens of the urinary tract.

The aforementioned compositions allow to attack Escherichia coli successfully even when it is protected by a biofilm.

Fosfomycin will preferably be used as the trihydroxymethylaminomethane salt or as the sodium salt.

Preparation of the compositions of the invention is performed according to conventional methods using additives that are known and used in the pharmaceutical field.

Depending on the route of administration selected, they can be in the form of injectable vials or solid compositions for oral use, for example capsules, tablets and effervescent tablets.

When it is desired to treat Escherichia coli infections, the preferred route of administration will be oral or by injection.

The compositions of the invention can be used in a preventive treatment.

The compositions can also be used as co-adjuvants in a conventional antibiotic therapy when this proves to be of poor efficacy owing to the protective action of the biofilm on the microorganism.

The effective doses of fosfomycin are of from 200 to 3000 mg/day to be taken in one or more administrations.

The effective doses of NAC are of from 128 to 2000 mg/day to be taken in one or more administrations.

The aforementioned dosages are the preferred ones because they are similar to the dosages currently administered in relation to known drugs based on fosfomycin and NAC, respectively. However, the practitioner may vary dosages and administration times without departing from the scope of the present invention.

The following examples are now given with the aim of better describing the present invention without limiting it in any way.

The effects of NAC and fosfomycin on the biofilm produced by Escherichia coli have been evaluated on four strains designated 169, 255, 1263 and 1293, from the Microbiological Collection of the Genua University.

### EXAMPLE 1

### Formation and Quantification of the Biofilm

Formation of the biofilms was measured spectrophotometrically in U-bottom polystyrene microplates (Corning Incorporated, NY).

In more detail, the effect of NAC on the formation of biofilms was evaluated as follows: overnight cultures in Tryptic Soy Broth (TSB) with addition of glucose (0.25%). (TSBG) were diluted 1:100 in TSBG. These suspensions (100 μl each) were added to suitable dilutions of NAC at concentrations ranging from 8 mg/ml to 0.007 mg/ml respectively. After incubation at 37° C. for 24 hours, the microplates were washed three times with 10 mM phosphate buffer pH 7.4 (PBS), treated with Bouin fixative and then stained with Crystal Violet (0.01%).

The biofilm adhering to the walls of the microplates was collected via washing with PBS, suspended in 10% sarbosyl and measured spectrophotometrically at 492 nm.

The same method was used for calculating the residual quantity of biofilm after treatment with NAC and fosfomycin, alone or associated, on Escherichia coli. For this purpose, biofilms with two different degrees of maturity: initial (5 hours) and fully consolidated (48 hours) were exposed to appropriate concentrations of NAC (from 8 mg/ml to 0.007 mg/ml) with addition of fosfomycin at the concentrations attainable in vivo in the urine.

At the same time the biofilms treated in this way were submitted to sonication in order to disperse the cells that were still adhering, which were seeded on rich medium after suitable dilution for calculating the viable units that survived the various treatments.

### EXAMPLE 2

### Effect of NAC on the Formation and Disruption of Biofilms and on Bacterial Viability

In all the strains of Escherichia coli tested, the various concentrations of NAC used, added to the cultures, did not cause appreciable changes in bacterial growth, but they did cause reductions in biofilm ranging from 73.8 to 35.9% and greater than 50% in three strains out of four.

A reduction greater than 50% (51.7%, 64.0%, 58.4%) was recorded on the initial bibfilms of Escherichia coli; similarly, with the mature biofilms the reduction is greater than 50% in three strains out of four (64.3%, 60.2%. and 67.7%), and in Escherichia coli 169 the reduction was equal to 25.2%.

The activity of NAC at the maximum concentration used on the cells of the biofilm produced decreases in CFU/ml ranging from 14.6% to 71.4% and from 9.1% to 54.2% respectively for biofilms at the stage of formation and for the consolidated biofilms.

### EXAMPLE 3

### Effect of Fosfomycin used Individually on the Disruption of Biofilms and on Bacterial Viability

Fosfomycin at the maximum concentration used (2000 mg/l) caused decreases in the quantity of biofilm ranging from 40% to 56.9% and from 41.5% to 49.3% respectively for the young and mature biofilms.

Even at 12.8 mg/l, fosfomycin produced reductions in polysaccharide material ranging from 30% to 48.1% and from 27.5% to 44.7%.

At the maximum concentration used (2000 mg/l), fosfomycin caused reductions in viable uriits ranging from 98.0% to 99.5% in the initial biofilms and ranging from 36% to 85.7% in the fully consolidated biofilms.

Reductions in CFU/ml less than 1 logarithm were caused by 128 mg/l of fosfomycin.

### EXAMPLE 4

### Effect of Fosfomycin Associated with NAC on Disruption of the Biofilms and on Bacterial Viability

In the strains of Escherichia coil tested, for both the concentrations used, fosfomycin exhibited the greatest disruptive effect combined with NAC (2 mg/ml), fosfomycin at a concentration of 2000 mg/l in combination with NAC (2 mg/ml) caused a decrease in initial biofilms ranging from 66.6% (Escherichia coli 1263) to 80.1% (Escherichia coli 169) and on the fully consolidated biofilms from 60.2% (Escherichia coli 1293) to 73.4% (Escherichia coli 255).

These reductions proved to be greater than those obtained with fosfomycin (from 50.3% to 74.4%) and NAC (from 39% to 58.8%) tested separately.

However, on increasing the concentrations of NAC to 4-8 mg/ml, the percentage reductions in biofilm disruption decrease. Thus, for the initial biofilms the reduction ranges from 46.2% (Escherichia coil 1263) to 53.5% (Escherichia coli 1293), and for the fully consolidated biofilms from 46.8% (Escherichia coli 1263) to 55.8% (Escherichia cofi 1293).

On the cells, the reduction of colony forming units CFU/ml was less than 3 (99.9%) and 2 logarithms (99%) for the combinations: fosfomycin 2000 mg/l and NAC 2 mg/ml, fosfomycin 2000 mg/l and NAC 0.5 mg/ml, fosfomycin 2000 mg/l and NAC 0.007 mg/ml respectively for immature and consolidated biofilms. Reductions of CFU/ml close to one logarithm were observed when fosfomycin at 128 mg/l was added to the same concentrations of NAC.

1-8. (canceled)

9. A pharmaceutical composition comprising N-acetylcysteine and fosfomycin, wherein said pharmaceutical composition is capable of inhibiting or disrupting one or more biofilms produced by one or more pathogens of the urinary tract.

10. The pharmaceutical composition as claimed in claim 9, wherein said one or more pathogens is Escherichia coli.

11. The pharmaceutical composition as claimed in claim 9, wherein said fosfomycin is in the form of a sodium salt or trihydroxymethylaminomethane salt.

12. A method of inhibiting or disrupting one or more biofilms produced by one or more pathogens of the urinary tract, said method comprising:
administering fosfomycin to a patient in need thereof in an amount effective to inhibit or disrupt one or more biofilms produced by one or more pathogens of the urinary tract.

13. The method as claimed in claim 12, wherein said fosfomycin is administered in an amount of 200 to 3000 mg/day.

14. The method as claimed in claim 12, further comprising administering N-acetylcysteine.

15. The method as claimed in claim 14, wherein said N-acetylcysteine is administered in an amount of 128 to 2000 mg/day.

16. The method as claimed in claim 12, wherein said one or more pathogens is Escherichia coli.

17. The method as claimed in claim 12, wherein said fosfomycin is in the form of a sodium salt or trihydroxymethylaminomethane salt.

18. A process for preparing a pharmaceutical composition, said process comprising:
preparing said pharmaceutical composition which comprises fosfomycin in a form suitable for injection or for oral administration.

19. The process as claimed in claim 18, wherein said pharmaceutical composition is capable of inhibiting or disrupting one or more biofilms produced by one or more pathogens of the urinary tract.

20. The process as claimed in claim 18, wherein said pharmaceutical composition further comprises N-acetylcysteine.

21. The process as claimed in claim 18, wherein said one or more pathogens is Escherichia coli.

22. The process as claimed in claim 18, wherein said fosfomycin is in the form of a sodium salt or trihydroxymethylaminomethane salt.
